(19) United States
(12) Patent Application Publication
(10) Pub. No.: US 2024/0042209 A1
(43) Pub. Date: Feb. 8, 2024

(54) MULTI IMPLANTABLE DEVICE COMMUNICATION AND CHARGING SYSTEM

(71) Applicant: Shiratronics, Inc., Brooklyn Park, MN (US)

(72) Inventors: Charles W. Steidl, Edina, MN (US); Alan H. Smythe, White Bear Lake, MN (US); Douglas W. Brown, Shakopee, MN (US); Jared R. Johnson, Wyoming, MN (US); James L. Kruse, New Brighton, MN (US); Andrew J. Sowada, St. Paul, MN (US)

(21) Appl. No.: 18/224,191

(22) Filed: Jul. 20, 2023

Related U.S. Application Data
(60) Provisional application No. 63/370,588, filed on Aug. 5, 2022.

(51) Int. Cl. A61N 1/36

(52) U.S. Cl. CPC A61N 1/36125

(57) ABSTRACT

A system may include at least two coil circuit branches, a driver, and a controller. Each of the at least two coil circuit branches may include a coil and a switch. The at least two coil circuit branches may be connected in parallel. The driver may be connected to the at least two coil circuit branches that are connected in parallel. The driver may be configured to drive the coils in the at least two coil circuit branches. The controller may be configured to independently control the switch in each of the at least two coil circuit branches to independently control whether the driver is electrically connected to drive the coil in the corresponding coil circuit branch. Some system embodiments include a dedicated driver for each of the coil circuit branches, and a controller configured to independently control the drivers to independently control whether individual coils are driven.

### PRIORITY

This application claims the benefit of priority to U.S. Provisonal Patent Application Ser. No. 63/370,588, filed Aug. 5, 2022, which is incorporated by reference herein in its entirety.

### TECHNICAL FIELD

This document relates generally to medical systems, and more particularly, but not by way of limitation, to systems, devices, and methods for charging and otherwise interacting with medical devices.

### BACKGROUND

Medical devices may include devices configured to deliver a therapy, such as but not limited to an electrical or drug therapy and /or to sense physiological or functional parameters or other health-related data. The medical devices may include external wearable devices and may include implantable devices. Each of these devices may include a rechargeable battery. Implantable devices configured to deliver an electrical therapy are a specific example of a medical device, and implantable neurostimulators are a specific example of implantable electrical therapy devices. A fully head-located implantable peripheral neurostimulation system, having at least two implantable devices, designed for the treatment of chronic head pain is a specific example of a system with more than one implantable device.

Neurostimulation systems may include a rechargeable battery or other power source such as a primary cell battery, an antenna coil, and circuitry to control the neurostimulation. The systems may include one or more implantable devices configured to connect with an external unit to perform various functions such as recharging the rechargeable battery, diagnostically evaluating the implantable device(s), and programming the implantable device(s).

There are a number of challenges for a system that includes multiple implantable devices and an external device. Coils may be used for communication and power transfer. The coils in the external device may be referred to as transmit coils (Tx coils) as it is the coil which is transmitting energy, and the implantable device coils may be referred to as receive coils (Rx coils) as they receive the energy transmitted from the Tx coils. Charging and communication may function well if both implantable devices are of similar alignment (at similar distances from the Tx coils and are presenting similar loads to the Tx coils. However, charging and communication breaks down if the system becomes unbalanced, such as when one implantable device is close to the Tx coil and one is far away or when one implantable device is charging. In an unbalanced system, one implantable device presents a large load and the other implantable device presents a small load. For example, if implantable Device A is close to a Tx coil and implantable Device B is far away, then communication with Device B may not be possible as Device A may absorb most of RF (radio frequency) energy for transmission of data leaving little energy for Device B to receive. Even more challenging is the transmission of data from Device B to the external device (e.g., charger). The changes in load of Device B to transmit data to the external device are masked by the large amount of power received by Device A. The overall change in power coupling does not change much, so the Tx coil current does not change much and cannot be observed by the external device. Additionally, charging multiple implantable devices is also challenging, particularly when Device A is close to the Tx coil and Device B is far away. For example, if Device A is fully charged, but Device B still requires charging, then it is difficult to steer the power to Device B. Device A can eliminate some of the power it absorbs by reducing the Q (Quality Factor) or increasing the loss factor of its receive coil by dissipating the RF rectified power through a shunt load to ground. However, Device A will still pick up power and greatly reduce overall charging efficiency. It is also challenging to detect proper alignment of the implantable devices because, if one device is much closer than the other, then the closer implantable device masks the changes in coil current by the device farther away. The alignment of the device farther away cannot be determined.

Improved techniques are desired for systems with multiple implantable devices.

### SUMMARY

An example (e.g., “Example 1”) of a system may include at least two coil circuit branches, a driver, and a controller. Each of the at least two coil circuit branches may include a Tx coil and a switch. The at least two coil circuit branches may be connected in parallel. The driver may be connected to the at least two coil circuit branches that are connected in parallel. The driver may be configured to drive the Tx coils in the at least two coil circuit branches. The controller may be configured to independently control the switch in each of the at least two coil circuit branches to independently control whether the driver is electrically connected to drive the Tx coil in the corresponding coil circuit branch.

In Example 2, the subject matter of Example 1 may optionally be configured such that, for each of the at least two coil circuit branches, the switch is connected in series with the Tx coil.

In Example 3, the subject matter of any one or more of Examples 1-2 may optionally be configured such that each of the at least two coil circuit branches further includes a resonant circuit.

In Example 4, the subject matter of Example 3 may optionally be configured such that, for each of the at least two coil circuit branches, the switch, the resonant circuit and the Tx coil are connected in series.

In Example 5, the subject matter of any one or more of Examples 1-4 may optionally be configured such that at least two implantable devices are configured to be charged using the at least two coil circuit branches.

In Example 6, the subject matter of Example 5 may optionally be configured such that the system is configured to independently control the switch in each of the at least two coil circuit branches to independently control which one or more of the at least two implantable devices are being charged.

In Example 7, the subject matter of Example 6 may optionally be configured such that when charging any one or more of the at least two implantable devices, the system is configured to determine and respond to an over-temperature event and respond to the determined over-temperature event by controlling the switch to prevent the driver from driving one or more of the Tx coils until the system determines that the over-temperature event has ended.

In Example 8, the subject matter of any one or more of Examples 6-7 may optionally be configured such that the system is configured to simultaneously charge the at least two implantable devices, to determine when charging for one of the at least two implantable devices is completed, to control the switch to prevent the driver from driving the Tx coil for one of the at least two coil circuit branches that corresponds to the one of the at least two implantable devices with charging completed, and continuing to charge one or more other devices from the at least two implantable devices.

In Example 9, the subject matter of any one or more of Examples 1-8 may optionally be configured such that the controller is configured to receive feedback from each of the at least two implantable devices using uplink communication, and to detect coil alignment using the received feedback.

In Example 10, the subject matter of any one or more of Examples 1-9 may optionally be configured such that the controller is configured to receive feedback via an uplink from each of the at least two implantable devices, and to detect charging status using the received feedback.

In Example 11, the subject matter of any one or more of Examples 1-10 may optionally be configured such that the controller is configured to receive feedback via an uplink from each of the at least two implantable devices, the received feedback is based on a PWRIN signal in each of the least two implantable devices, and the PWRIN signal corresponds to a rectified voltage.

In Example 12, the subject matter of any one or more of Examples 1-11 may optionally be configured such that the at least two coil branches include a first coil branch corresponding to a first implantable device and a second coil branch corresponding to a second implantable device, and the controller is configured to determine coil alignment by: connecting the first coil branch to the driver, using the driver to drive the first coil branch to produce a first fixed charge field, and receiving a first signal from the first implantable device indicative of electrical energy transfer from the external device; and connecting the second coil branch to the driver, using the driver to drive the second coil branch to produce a second fixed charge field, and receiving a second signal from the second implantable device indicative of electrical energy transfer from the external device.

In Example 13, the subject matter of any one or more of Examples 1-12 may optionally be configured such that the controller is configured to implement a recharge session by using the driver to drive the two or more coil circuit branches to produce a charge field for recharging a corresponding two or more implantable devices, receiving a signal that charging for one or more of the at least two implantable devices should be stopped, responding to the signal by disconnecting one or more of the two or more coil circuit branches that correspond to the one or more of the at least two implantable devices for which charging should be stopped, and continuing to charge one or more other devices from the at least two implantable devices.

In Example 14, the subject matter of Example 13 may optionally be configured such that the signal indicates that the one or more of the at least two implantable devices are fully charged.

In Example 15, the subject matter of Example 13 may optionally be configured such that the signal indicates that there is a temperature event associated with the charging of the one or more of the at least two implantable devices.

In Example 16, the subject matter of Example 15 may optionally be configured such that the temperature event is determined using at least one temperature sensor on the external device.

In Example 17, the subject matter of Example 15 may optionally be configured such that the temperature event is determined using at least one temperature sensor on the one or more of the at least two implantable devices.

In Example 18, the subject matter of any one or more of Examples 15-17 may optionally be configured such that the one or more of the at least two coil circuit branches are temporarily disconnected in response to the temperature event until the temperature event is over.

Example 19 includes subject matter (such as a method, means for performing acts, machine readable medium including instructions that when performed by a machine cause the machine to performs acts, or an apparatus to perform). The subject matter may be performed using an external device having a driver and at least two switches corresponding to at least two transmit (Tx) coils, wherein the at least two Tx coils correspond to at least two implantable devices, wherein each of the at least two switches are configured for electrically connecting a corresponding one of the at least two Tx coils to the driver. The subject matter may include independently controlling each of the at least two switches to cause one or more of the at least two Tx coils to be electrically-connected coils to the driver; and using the driver to drive the one or more electrically-connected Tx coils to the driver.

In Example 20, the subject matter of Example 19 may optionally be configured to further include receiving a signal from each of one or more implantable devices corresponding to the one or more electrically-connected Tx coils, wherein the received signal is indicative of electrical energy transfer from the external device to the corresponding implantable device.

In Example 21, the subject matter of Example 20 may optionally be configured to further include recharging one or more of the at least two implantable devices, wherein the received signal is indicative of a full charge state, the method further comprising responding to the signal by controlling a corresponding one of the at least two switches to disconnect a corresponding one of the at least two Tx coils from the driver.

In Example 22, the subject matter of any one or more of Examples 20-21 may optionally be configured to further include determining coil alignment between a selected one of the at least two Tx coils and a receive (Rx) coil in a corresponding one of the implantable devices. The subject matter may include driving the selected one of the at least two Tx coils to generate a fixed charge field. The signal may be indicative of the electrical energy transfer from the external device using the fixed charge field. The external device may be configured to determine coil alignment using the signal.

Example 23 includes subject matter (such as a method, means for performing acts, machine readable medium including instructions that when performed by a machine cause the machine to performs acts, or an apparatus to perform). The subject matter may be performed by an external device to recharge two or more implantable devices where the external device has a driver configured to drive two or more electrically-connected transmit (Tx) coils corresponding to the two or more implantable devices. The subject matter may include using the driver to drive the two or more electrically-connected Tx coils to produce a charge field for recharging the two or more implanted devices, receiving a signal that charging for one or more of the at least two implantable devices should be stopped, responding to the signal by disconnecting one or more of the two or more Tx coils that correspond to the one or more of the at least two implantable devices for which charging should be stopped, and continuing to charge one or more other devices from the at least two implantable devices.

In Example 24, the subject matter of Example 23 may optionally be configured such that the signal indicates that the one or more of the at least two implantable devices are fully charged.

In Example 25, the subject matter of Example 23 may optionally be configured such that the signal indicates that there is a temperature event associated with the charging of the one or more of the at least two implantable devices.

In Example 26, the subject matter of Example 25 may optionally be configured such that the temperature event is determined using at least one temperature sensor on the external device.

In Example 27, the subject matter of Example 25 may optionally be configured such that the temperature event is determined using at least one temperature sensor on the one or more of the at least two implantable devices.

In Example 28, the subject matter of any one or more of Examples 25-27 may optionally be configured such that the one or more of the two or more Tx coils is temporarily disconnected in response to the temperature event until the temperature event is over.

Example 29 includes subject matter (such as a method, means for performing acts, machine readable medium including instructions that when performed by a machine cause the machine to performs acts, or an apparatus to perform). The subject matter may be performed by an external device having a driver configured to drive at least a first transmit (Tx) coil and a second Tx coil. The subject matter may include determining coil alignment between the first Tx coil and a receive (Rx) coil for a first implantable device, including connecting the first Tx coil to the driver, using the driver to drive the first Tx coil to produce a first fixed charge field, and receiving a first signal from the first implantable device indicative of electrical energy transfer from the external device. The subject matter may include determining coil alignment between the second Tx coil and a Rx coil for a second implantable device, including connecting the second Tx coil to the driver, using the driver to drive the second Tx coil to produce a second fixed charge field, and receiving a second signal from the second implantable device indicative of electrical energy transfer from the external device.

In Example 30, the subject matter of Example 29 may optionally be configured such that the coil alignment between the first Tx coil and the Rx coil for the first implantable device is determined when the coil alignment between the second Tx coil and the Rx coil for the second implantable device is determined. The coil alignment may be performed concurrently, but does not necessarily have to start at the same time and does not necessarily have to stop at the same time.

In Example 31, the subject matter of Example 29 may optionally be configured such that the coil alignment between the first Tx coil and the Rx coil for the first implantable device is determined at a different time than when the coil alignment between the second Tx coil and the Rx coil for the second implantable device is determined.

Example 32 may be a system that includes at least two coil circuit branches, at least one driver and a controller. Each of the at least two coil circuit branches may include a transmit (Tx) coil. The at least one driver may be connected to the at least two coil circuit branches to drive the Tx coils. The controller may be configured to independently control whether individual Tx coils are driven.

In Example 33, the subject matter of Example 32 may optionally be configured such that the at least one driver includes a dedicated driver for each one of the at least two coil circuit branches. The controller may be configured to independently control the drivers to independently control whether individual Tx coils are driven.

In Example 34, the subject matter of Example 32 may optionally be configured such that each of the at least two coil circuit branches includes a controller-controlled switch, and the at least two coil circuit branches are in parallel.

In Example 35, the subject matter of Example 32 may optionally be configured such that the at least two coil circuit branches are configured to be connected in a series connected circuit, and the controller is configured to independently control whether individual Tx coils are driven by controlling whether each of the at least two coil circuit branches are in the series connected circuit.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIGS. 1A-1B illustrate a system that includes implantable device(s) and an external device configured for use to communicate with and charge the implantable device(s).

FIG. 2A depicts two implanted devices with leads to cover both sides of the head with one on the left side of the head and the other on the right side of the head, and FIG. 2B illustrates a charging/communication headset disposed about the cranium.

FIG. 3 illustrates, by way of example and not limitation, an embodiment of an external charging system configured to individually control whether transmit coils are driven using parallel coil circuit branches selectively connected to a single driver via switches controlled by the controller.

FIG. 4 illustrates, by way of example and not limitation, an embodiment of an external charging system that includes a cable-connected headset.

FIG. 5 illustrates, by way of example and not limitation, an embodiment of an external charging system that includes a cable-connected headset.

FIG. 6 illustrates, by way of example and not limitation, an embodiment of an implantable device.

FIG. 7 illustrates, by way of example and not limitation, an embodiment of an implantable device that uses negative peak detection to receive telemetry and a half-wave rectifier for power.

FIG. 8 illustrates, by way of example and not limitation, a block diagram of an device.

FIG. 9 illustrates, by way of example and not limitation, a method that may be performed using an external device having a driver and at least two switches corresponding to at least two Tx coils that correspond to at least two implantable devices.

FIG. 10 illustrates, by way of example and not limitation, a method for charging two or more implantable devices.

FIG. 11 illustrates, by way of example and not limitation, a method for determining coil alignment with implantable devices.

FIG. 12 illustrates, by way of example and not limitation, a method for charging two or more implantable devices while monitoring and accommodating temperature events.

FIG. 13, for example, illustrates separate drivers, individually controlled by a controller, to drive separate Tx coil.

FIG. 14, for example, illustrates series-connected coils circuit branches selectively connected to a single driver via switches controlled by a controller.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Various embodiments may be used to efficiently, reliably, and safely charge, communicate, and determine alignment with one or more implant devices wirelessly. During communication and/or alignment, individual headset coils may be electrically isolated to minimize any impact of parallel resonance on inductive communication. During recharge, all headset coils charging an implant device are connected in parallel.

Charging and communication may be achieved in a system with multiple implantable devices without isolation when both IPGs are of similar alignment (at similar distances from the transmit (Tx) coils) and are both presenting similar loads to the Tx coils. However, as identified above, charging and communication breaks down if the system becomes unbalanced such as may occur if one implanted device is close to the Tx coil and one is far away, or when one implanted device is charging and presents a large load on its TX coil and the other implanted device is done charging and presents a small load to its Tx coil.

Various embodiments of the present subject matter may isolate power to each Tx coil or drive them concurrently. Thus, the present subject matter is capable of accommodating different Tx coil to Rx coil alignment scenarios and implanted device modes of operation to charge and communicate with multiple implanted devices safely, efficiently, and reliably. For example, in the situation where Device A is much closer to its Tx coil than Device B, transmit power may be isolated to just the Tx coil for Device B and may be disconnected from the Tx coil for Device A. By isolating power to the Tx coil B to communicate only with Device B, the communication can be accommodated over a larger distance range that is not affected by the alignment of Device A. Additionally, charging of implanted Device B can be completed and performed much more efficiently. When both implantable devices (or two or more implantable devices for system that have more than two implantable devices) need charging, then the external device can enable both Tx coils and charging can occur concurrently. When one of the implantable devices completes charging, then the Tx coil for the charged implanted device may be disconnected, and the wireless power can be isolated to the other device(s) to complete its charging. By charging both concurrently and then isolating, the overall charging time may be reduced. If the system could only accommodate enabling one Tx coil at a time, then the total time to charge two implanted devices may almost be double compared to a system that could charge them concurrently.

Thus, various embodiments of the present subject matter may provide an external device that can charge two devices simultaneously or isolate charging to each device. This flexibility enables efficient charging in all scenarios. For example, if both devices need charging and are aligned similarly then both can be charged at the same time. Then when one device finishes charging then wireless energy can be isolated to the other device to complete charging in the shortest time possible and in the most efficient way for the charger. The external device may communicate with and charge the battery of Device B when Device A completes battery charging by isolating power to Device B for charging and communication. The external device may communicate with Device B while it is far from the Tx coil and Device A is close to a Tx coil by disconnecting the Tx coil for Device A and isolating power to the Tx coil for Device B. The external device may charge multiple devices (e.g., Device A and Device B) and respond when one device (Device A) has an over-temperature event by disconnecting the Tx coil for Device A and isolating power to Device B for charging and periodically check the temperature of Device A. An over-temperature event may be an event during which a temperature measurement is over a temperature threshold, such as may occur during recharging of a device, and may be defined as an event where a temperature and duration of generated heat exceeds a threshold such as CEM43 dose thresholds as discussed in U.S. Provisional Application No. ______ (Attorney Docket 5467.016PRV), entitled “Thermal Management of Medical Devices”, and filed on the same date as the present application, which is herein incorporated by reference in its entirety. Once the temperature of Device A reduces below the over-temperature limit, the charger may resume charging of Device A. The external device may detect proper alignment of the Tx coil to the Rx coil of the implantable device. The implantable device may provide direct/active feedback from the implantable device for a measure of alignment compared to other approaches that use passive measurements (Tx coil current, or reflective impedance) that do not utilize feedback from the implantable device.

By way of example, this disclosure discusses a fully head located implantable peripheral neurostimulation system designed for the treatment of chronic head pain. The system may be configured to provide neurostimulation therapy for chronic head pain, including chronic head pain caused by migraine and other headaches, as well as chronic head pain due other etiologies. For example, the system may be used to treat chronic head and/or face pain of multiple etiologies, including migraine headaches; and other primary headaches, including cluster headaches, hemicrania continua headaches, tension type headaches, chronic daily headaches, transformed migraine headaches; further including secondary headaches, such as cervicogenic headaches and other secondary musculoskeletal headaches; including neuropathic head and/or face pain, nociceptive head and/or face pain, and/or sympathetic related head and/or face pain; including greater occipital neuralgia, as well as the other various occipital neuralgias, supraorbital neuralgia, auriculotemporal neuralgia, infraorbital neuralgia, and other trigeminal neuralgias, and other head and face neuralgias.

The system may include two implantable devices bilaterally implanted on the right and left sides of the patient's head. However, the present subject matter is not limited to such systems, as those of ordinary skill in the art would understand, upon reading and comprehending this disclosure, how to implement the teachings herein with other systems with two or more rechargeable devices such as two or more rechargeable medical devices that are implantable or wearable.

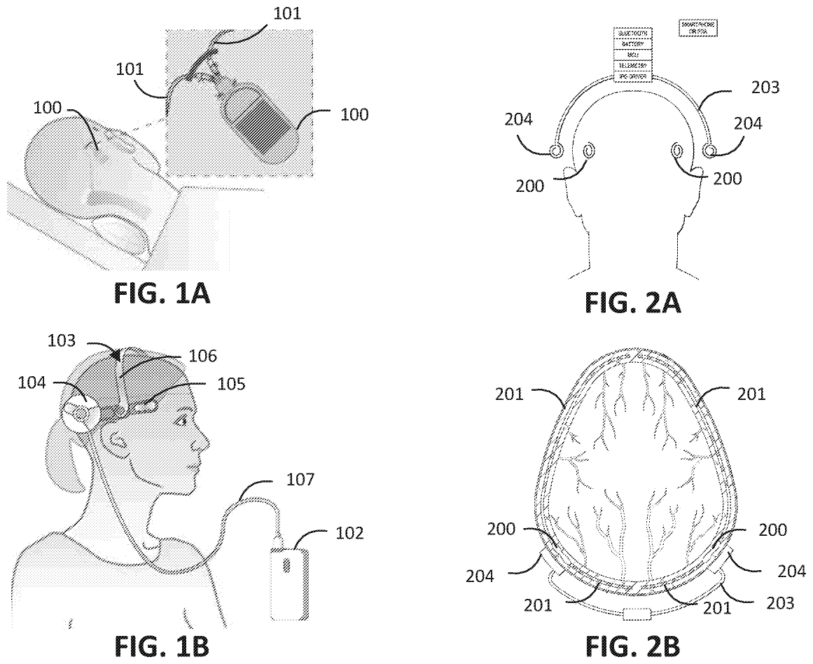

FIGS. 1A-1B illustrate a system that includes implantable device(s) and an external device configured for use to communicate with and charge the implantable device(s). FIG. 1A illustrates an implantable device 100 implanted beneath the skin and over a patient's cranium. The device 100 is illustrated as being implanted behind and above the ear. The implantable device may include one or more leads 101 that may be subcutaneously tunneled to a desired neural target. Each lead may include one or more electrodes. The number of electrodes and spacing may be such as to provide therapeutic stimulation over any one or any combination of the supraorbital, parietal, and occipital region substantially simultaneously. The implantable device 100 may be configured to independently control each electrode to determine whether the electrode will be inactive or configured as an electrode or an anode. One or more electrodes on the lead(s) may be configured to function as an anode, and one or more electrodes on the lead may be configured to function as a cathode. For example, bipolar neuromodulation may be delivered using one or more anodes and one or more cathodes on the lead(s). A clinician may program the electrode configurations to provide a neuromodulation field that captures a desired neural target for the therapy.

FIG. 1B illustrates an external device 102 and headset 103 configured for use to communicate with and/or charge the implantable device(s) 100. The headset 103 may include an Tx coil 104, and the headset 103 may be configured to position the Tx coil over an implantable device. For example, the headset 103 may include an adjustable frame 105 on each side of the head that can rotate about a point on a main headset frame 106, and slide in or away from the main headset frame 106. These adjustable frames may be used to position the Tx coils 104 over the implantable devices 100. The external device 102 may be electrically connected to the headset 103 via a cable 107. The external device 102 may communicate and/or charge the headset 103 using the cable 107. The external device 102 may use the headset 103 to communicate (e.g., program or other communication) with the implantable device(s) 100. In some embodiments, the external device 102 may be wirelessly connected to the headset 103. The headset may be configured to wirelessly receive power from the external device and to transfer power from the Tx coil to the implanted device(s).

FIG. 2A depicts two implanted devices 200 with leads 201 to cover both sides of the head with one on the left side of the head and the other on the right side of the head, and FIG. 2B illustrates a charging/communication headset 203 disposed about the cranium. The headset 203 may include right and left coupling coil enclosures, respectively that contain Tx coils for coupling to the respective Rx coils in the implants. The coil enclosures interface with a main charger/processor body which contains processor circuitry and batteries for both charging the internal battery in the implantable devices 200 and also communicating with the implanted devices. Thus, in operation, when a patient desires to charge their implanted devices 200, all that is necessary for some embodiments is to place the headset about the cranium with the Tx coils 204 in close proximity to the respective implanted devices 200. In some embodiments, such placement may automatically initiate charging; whereas in other embodiments, the user may initiate charging using an external device. When the headset 203 is worn by a patient, the Tx coils 204 are placed in proximity to the corresponding Rx coil in each respective body-implanted implantable device 200. The exemplary headset 203 may include an implantable device driver, telemetry circuitry, a microcontroller (MCU), a battery, and a Bluetooth wireless interface. The headset 203 may also communicate with a personal device such as a smartphone or tablet, for monitoring and/or programming operation of the two implantable devices. In various embodiments, some or all of these elements may be contained in the external device instead of the headset.

The implantable device may include a rechargeable battery, an antenna (e.g., Rx coil), and an application specific integrated circuit (ASIC), along with the necessary internal wire connections amongst these related components, as well as to the incoming lead internal wires. These individual components may be encased in a can made of a medical grade metal and plastic cover. The battery may be connected to the ASIC via a connection that is flexible. The overall enclosure for the battery, antenna and ASIC may have a very low flat profile. The enclosure may have two sections, one section for housing the ASIC and one section for housing the battery. The sections of the housing may, but does not necessarily, provide separate enclosures for separate spaces. The antenna may be housed in either of the sections or in both sections. The use of the two sections and the flexible connection between the ASIC and the battery allows the implanted device to conform to the shape of the human cranium when subcutaneously implanted without securing such to any underlying structure with an external fixator.

The ASIC and lead may be configured to independently drive the electrodes using a neuromodulation signal in accordance with a predetermined program. The programmed stimulation may be defined using parameters such as one or more pulse amplitudes, one or more pulse widths and one or more pulse frequencies. Other parameters may be used for other defined waveforms, which may but does not necessarily use rectilinear pulse shapes. Once the program is loaded and initiated, a state machine may execute the particular program to provide the necessary therapeutic stimulation. The ASIC may have memory and be configured for communication and for charge control when charging a battery. Each of the set of wires and interface with the ASIC such that the ASIC individually controls each of the wires in the particular bundle of wires. Thus, each electrode may be individually controlled. Each electrode may be individually turned off, or as noted above, each electrode can be designated as an anode or a cathode. During a charging operation, the implanted device is interfaced with an external charging unit via the antenna (e.g., Rx coil) which is coupled to a similar antenna (e.g., Tx coil) in the external charging unit. Power management involves controlling the amount of charge delivered to the battery, the charging rate thereof and protecting the battery from being overcharged.

The ASIC may be capable of communicating with an external unit, typically part of the external charging unit, to exchange information. Thus, configuration information can be downloaded to the ASIC and status information can be retrieved. A headset or the like may be provided for such external charging/communication operation.

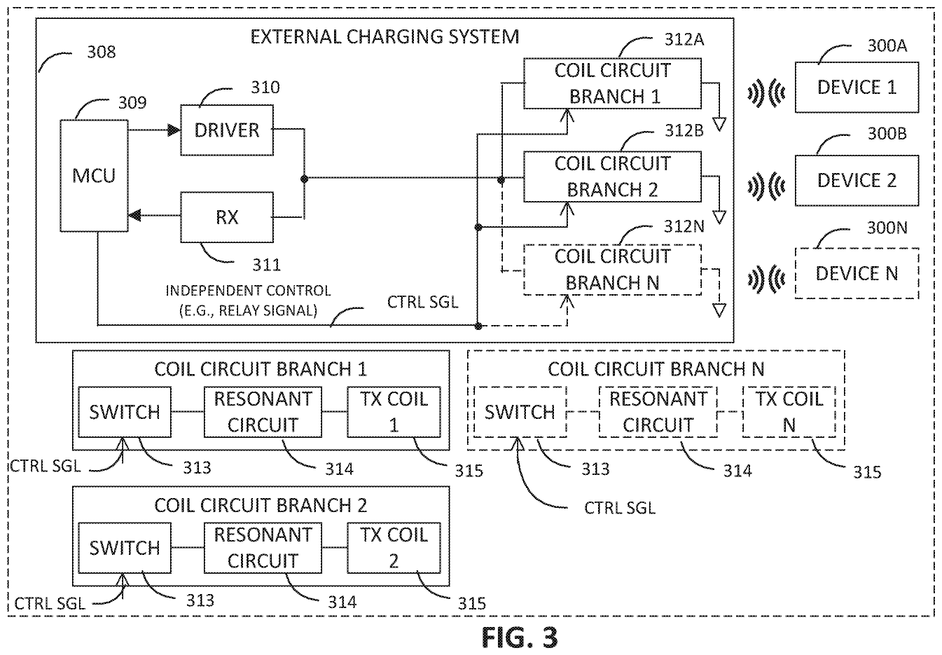

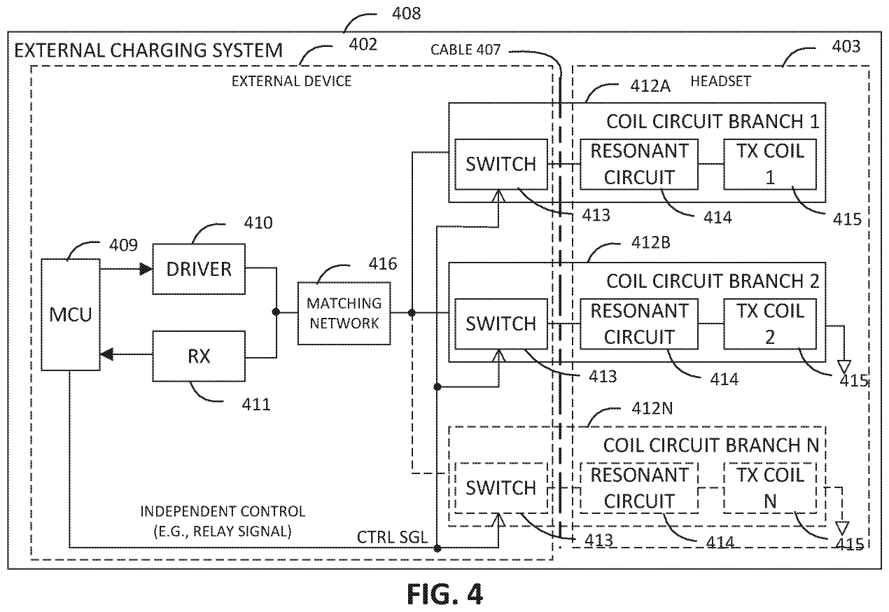

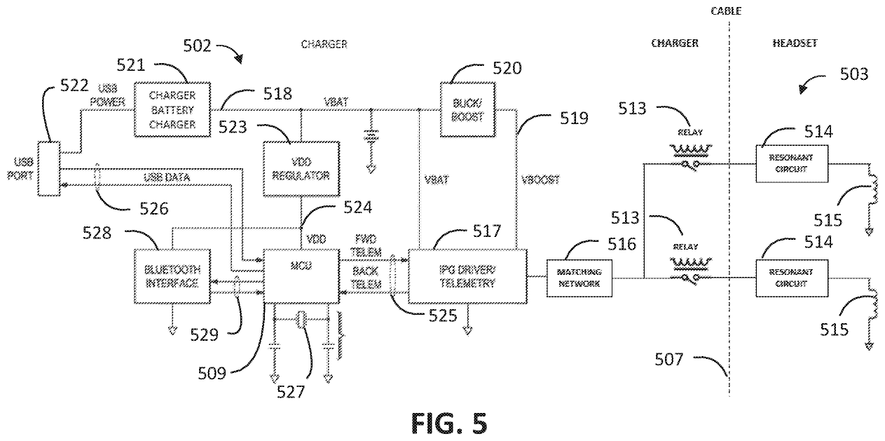

FIG. 3 illustrates, by way of example and not limitation, an embodiment of an external charging system configured to individually control whether transmit coils are driven using parallel coil circuit branches selectively connected to a single driver via switches controlled by the MCU. The external charging system 308 is configured to wirelessly charge at least two devices, and may be configured to wireless charge more than two devices (e.g., N devices), illustrated as Device 1 300A, Device 2 300B and Device N (300N). The external charging system 308 may take the form of the headset 103, 203 illustrated in FIGS. 1B, 2A-2B. The external charging system 308 may include a controller 309 (e.g., microcontroller or MCU), a driver circuit, or driver 310 and a receiver circuit, or receiver 311. The external charging system 308 may further include a first coil circuit branch 312A and a second coil circuit branch 312B. If configured to wirelessly charge N devices, the external charging system 308 may include N coil circuit branches, including an Nth coil circuit branch 312N. The coil circuit branches are connected in parallel. The devices may not be “paired” to the coil circuit branches, but rather any of the coil circuit branches may be positioned and used to wireless charge any of the devices. That is, they may be interchangeable and do not have to be paired. Each of the circuit branches 312A-312C may be selectively connected to the driver 310 and receiver 311 via a control signal (illustrated in drawings as “CTRL SGL”) from the controller 309. For example, each coil circuit branch may include a switch 313, a resonant circuit 314 and a Tx coil 315 connected in series. The switch 313 is configured to respond to the control signal from the controller 309 to selectively connect the Tx coil 315 for the circuit branch to the driver 310 and receiver 311. Thus, the controller 309 and switches cooperate to independently connect or disconnect the Tx coil from the driver 310 or receiver 311. By way of example, the switch 313 may be a relay switch. Other switch examples include transistors, TRIACs or other solid state switch devices. Thus, the external charging system 308 is capable of individually controlling which of the Tx coils in the parallel-connected, coil circuit branches is connected. The resonant circuit 314 creates a resonant frequency close to the drive frequency to maximize efficiency, and may also provide matching impedance to a cable such as a 75 Ohm cable (see cable 107 and FIGS. 4-5).

FIG. 4 illustrates, by way of example and not limitation, an embodiment of an external charging system that includes a cable-connected headset. The illustrated external charging system 408 includes a headset 403 connected to an external device 402, which may be similar to the headset 103 and external device 102 connected by cable 107 in FIG. 1B. The external charging system 408 may include a controller 409 (e.g., microcontroller or MCU), a driver circuit, or driver, 410 and a receiver circuit, or receiver, 411 in the external device 402. The external charging system 408 may further include parallel-connected coil circuit branches, including a first coil circuit branch 412A and a second coil circuit branch 412B. If configured to wirelessly charge N devices, the external charging system 408 may include N coil circuit branches, including an Nth coil circuit branch 412N. Each of the coil circuit branches may include a switch 413, such as a relay or other solid-state switch, resonant circuit 414 and coil 415, which may be connected in series with each other. The switches 413 are configured to respond to control signals (CTRL SGL) from the controller 409 to selectively connect the Tx coil 415 for the circuit branch to the driver 410 and receiver 411. The control signals may be sent over separate conductors between the controller 409 and each switch 413, or may be an addressable signal that may be addressed to each switch. Regardless, the controller 403 is able to individually control the switches 413. In the illustrated embodiment, the switches 413 are in the external device 402 and the resonant circuits 414 and Tx coils 415 are in the headset, with the cable 407 connecting the external device 402 to the headset 403. The illustrated external device 402 also includes a matching network 416 configured to match impedance to the cable 407, such as a 75 Ohm cable.

FIG. 5 illustrates, by way of example and not limitation, an embodiment of an external charging system that includes a cable-connected headset. The system of FIG. 5 may be a more specific example of the system illustrated in FIG. 4. The illustrated external charging system includes a headset 503 connected to an external device 502 (or “charger”) via a cable 507. The external device 502 includes an IPG (implantable Pulse Generator) driver and telemetry block 517 that drives two Tx coils 515, and which is powered by a battery voltage VBAT conveyed on node 518 by headset battery and an adjustable voltage VBOOST conveyed on node 519. A buck/boost circuit 520 receives the VBAT voltage on node 518 and generates the VBOOST voltage on node 519. The headset battery is charged by a headset battery charger 521 which receives USB power from USB port 522. A VDD regulator 523 also receives the VBAT voltage on node 518 and generates a VDD voltage (e.g., regulated to 3.0 volts) on node 524, which is generally used as a power supply voltage for certain circuitry within the headset.

A microcontroller (MCU) 509 provides general configuration control and intelligence for the headset 502, and communicates with the IPG driver and telemetry block 517 via a forward telemetry signal FWD TELEM (e.g., “downlink”) and a back telemetry signal BACK TELEM (e.g., “uplink”) via a pair of data lines 525. The MCU 509 may communicate with an external device (e.g., a smartphone, tablet), a controller, a diagnostic tester, a programmer, and the like) that is connected to the USB port 522 via a pair of USB data lines 526. The MCU 509 may be connected to an external crystal resonant tank circuit 527 for providing an accurate timing source to coordinate its various circuitry and data communication interfaces. A Bluetooth interface 528 may provide wireless interface capability to an external device, such as a smartphone or other host controller, and may be connected to the VDD voltage on node 524. The Bluetooth interface 528 may communicate with the MCU 509 using data/control signals 529. In general, MCU 509 may be used to store configuration information in an on-chip non-volatile memory for both the overall headset and charging system and also provide configuration information that can be transferred to one or more of the body-implanted devices. The overall operation of the headset may be that of a state machine, wherein the IPG driver/telemetry block 517 and the other surrounding circuitry, such as the buck/boost circuit 520 and the headset battery charger 521, all function as state machines, typically implemented within an ASIC. The MCU 509 may be activated when communication information is received that requires the MCU 509 to transfer configuration information to the body-implanted device or, alternatively, to configure the headset state machine. A state machine may be used for most functionality because it has lower power operation, whereas an instruction-based processor, such as the MCU 509 may require more power, it should be understood, however, that such a headset may use any type of processor, state machine or combinatorial logic device. In the illustrated embodiment, the relay switches 513 are in the external device 502 and the resonant circuits 514 and Tx coils 515 are in the headset, with the cable 507 connecting the external device 502 to the headset 503. The illustrated external device 502 also includes a matching network 516 configured to match impedance to the cable 507, such as a 75 Ohm cable.

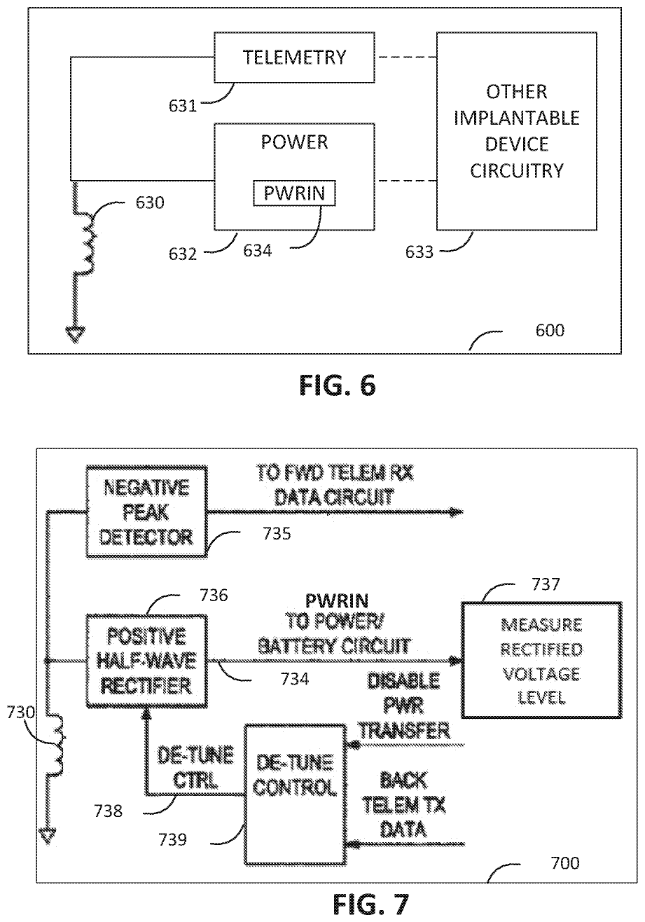

FIG. 6 illustrates, by way of example and not limitation, an embodiment of an implantable device. The illustrated implantable device 600 includes a Rx coil 630, telemetry circuitry 631 for use to communicate with the external system via the Rx coil, power circuitry 632 for receiving power form the external system via the Rx coil 630, and other implantable device circuitry 633. The power circuitry 632 generates a PWRIN 634 signal based on the energy received by the Rx coil 630. The device 600 may be configured to communicate data corresponding to the measured PWRIN 634 to the external device.

FIG. 7 illustrates, by way of example and not limitation, an embodiment of an implantable device that uses negative peak detection to receive telemetry and a half-wave rectifier for power. The implantable device of FIG. 7 may be a more specific example of the device illustrated in FIG. 6. The illustrated implantable device 700 includes a Rx coil 730, a negative peak detector 735, and a positive half-wave rectifier 736. The Rx coil 730 may be coupled to a negative peak detector block 735 for receiving downlink data and generating a respective downlink receive data signal. The Rx coil 730 coupled to the positive half-wave rectifier block 736 receives energy and generates a rectified voltage (PWRIN 734), which may be provided to a power/battery circuit. The illustrated device 700 may include a sensor 737 configured to measure the rectified voltage level of PWRIN 734. The device 700 may be configured to communicate data corresponding to the measured PWRIN 734 to the external device, which may use this data to control the charging and alignment routines. The positive half-wave rectifier block 736 may he responsive to a DE-TUNE signal for de-tuning the Rx coil 730 to inhibit transfer of energy from the external device. The implantable device may also include a de-tune control block 739 for generating the DE-TUNE control signal 738 responsive to a disable power transfer signal DISABLE PWR TRANSFER, and/or responsive to a bit-serial back telemetry (uplink) transmit data signal BACK TELEM TX DATA. In operation, the DISABLE PWR TRANSFER signal may be asserted when charging (or charge transfer) is complete or not desired, which asserts the DE-TUNE control signal to de-tune the Rx coil through the positive half-wave rectifier. During normal charging the DE-TUNE control signal may be asserted for each bit-position of the bit-serial BACK TELEM TX DATA signal corresponding to one of its two data states. Since de-tuning the positive half-wave rectifier in concert with the Rx coil inhibits energy transfer from the Tx coil to the Rx coil, the loading on the Tx coil is decreased. In the external device, the receiver circuit senses the change in peak current through the corresponding Tx coil as each serial data hit of the BACK TELEM TX DATA signal either tunes or de-tunes the Rx coil, and generates accordingly a back telemetry (uplink) receive data signal BACK TELEM RX DATA. If the DE-TUNE control signal is already asserted (e.g., because the DISABLE PWR TRANSFER signal is asserted to indicate charging/charge transfer is complete or not desired) when the charge receiving system desires to transmit uplink data, the DISABLE PWR TRANSFER signal may be briefly de-asserted to allow the BACK TELEM TX DATA signal to control the DE-TUNE control signal. Thus, the charge receiving system may still transmit uplink information irrespective of whether it is generally in a de-tuned state.

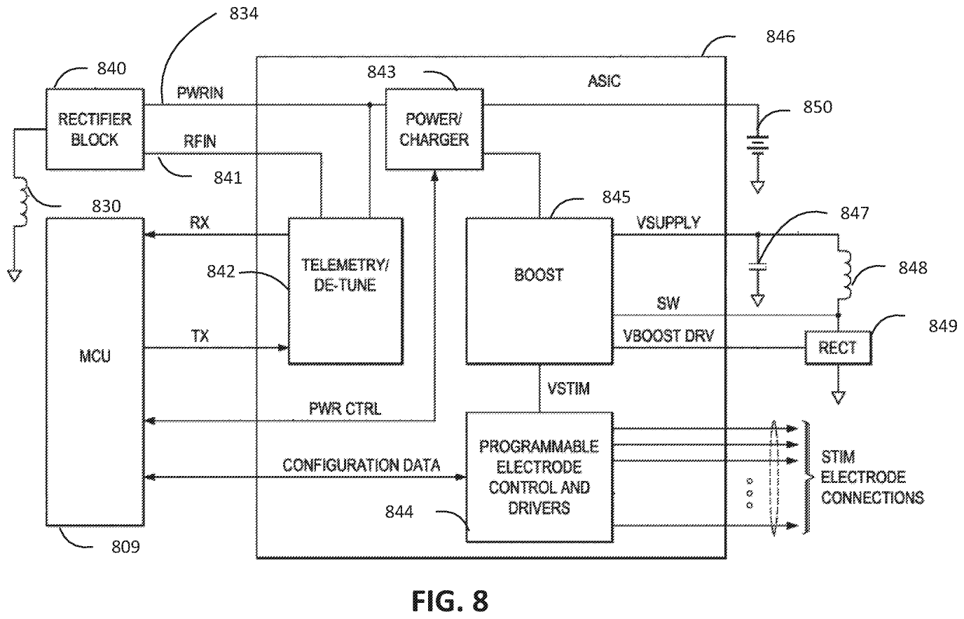

FIG. 8 illustrates, by way of example and not limitation, a block diagram of an implantable device 800. A Rx coil 830 is connected to a rectifier block 840 that generates a PWRIN 834 and an RFIN signal 841. Both the PWRIN signal 834 and the RFIN signal 841 are connected to a TELEMETRY/DE-TUNE block 842 that receives a forward telemetry or downlink signal (RFIN signal 841), and which interacts with the PWRIN de-tunes the Rx coil 830 to thereby communicate uplink information and/or disable further energy transfer to the Rx coil 830. The PWRIN node 834 is also connected to a power/charger block 843 which receives the PWRIN signal 834 to generate one or more internal voltages for the circuitry of the implantable device and for charging the battery 850.

A microcontroller (MCU) 809 provides overall configuration and communication functionality and communicates downlink (RX signal) and uplink (TX signal) information via a pair of data lines coupled to the telemetry block 842. The MCU 809 receives information from and provides configuration information to/from the power/charger block 843 via control signals PWR CTRL. A programmable electrode control and driver block (drivers) 844 generates electrical stimulation signals on each of a group of individual electrodes. An adjustable voltage generator circuit boost 845. which is coupled via signals VSUPPLY, SW, and VBOOST DRV to components external to the ASIC 846 (including capacitor 847, inductor 848, and rectifier block 849) provides a power supply voltage VSTIM to the drivers block 844.

The MCU 809 provides configuration information to the drivers block 844 via configuration signals CONFIGURATION DATA. In some embodiments, the power charger block 843, the telemetry block 842, the boost circuit 845, and the drivers block 844 are all implemented in a single application specific integrated circuit (ASIC) 846, although such is not required. In the overall operation, the ASIC 846 may function as a state machine that operates independently of the MCU 809. The MCU 809 may include nonvolatile memory for storing configuration data from the external control system to allow a user to download configuration data to the MCU 809. The MCU 809 may then transfer this configuration data to ASIC 846 in order to configure the state machine therein. In this manner, the MCU 809 does not have to operate to generate the driving signals on the electrodes which may reduce the power requirements. Other embodiments may implement one or more of these three functional blocks using a combination of multiple ASICs, off-the-shelf integrated circuits, and discrete components.

Battery charging (charge delivery) may be monitored and adjusted to provide the most efficient charging (charge delivery) conditions and limit unnecessary power dissipation. Preferable conditions for charging the battery may include a another PWRIN voltage on node 834 of approximately 4.5 V for most efficient energy transfer (with a minimum charging voltage of about 4.0 V). Also, it is particularly desirable to maintain a constant charging current into the battery in a battery charging operation during the entire charging time, even as the battery voltage increases as it charges. Preferably this constant charging current is about C/2, which means a charging current that is one-half the value of the theoretical current draw under which the battery would deliver its nominal rated capacity in one hour. To accomplish this, a variety of sensors and monitors (not shown) may be included within the device 800 to measure power levels, voltages (including the battery voltage itself), charging current, and one or more internal temperatures.

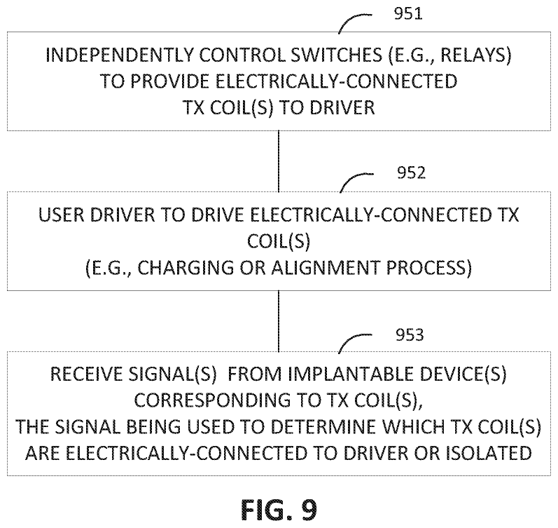

FIG. 9 illustrates, by way of example and not limitation, a method that may be performed using an external device having a driver and at least two switches corresponding to at least two Tx coils that correspond to at least two implantable devices. Each of the at least two switches may be configured for electrically connecting a corresponding one of the at least two Tx coils to the driver. The method may include independently controlling each of the at least two switches to cause one or more of the at least two Tx coils to be electrically-connected Tx coils to the driver 951 and using the driver to drive the one or more electrically-connected Tx coils to the driver 952. The method may further include receiving a signal from each of one or more implantable devices corresponding to the one or more electrically-connected Tx coils 953, wherein the received signal is indicative of electrical energy transfer from the external device to the implantable device. The received signal may be used by the external device to determine which of the Tx coil(s), if any, should be electrically-connected to the driver and which of the Tx coil(s), if any, should be isolated from the driver. That is, the external device may be configured to control the isolation of Tx coil(s) based on the received signal. For example, the method may include recharging one or more of the at least two implantable devices, and the received signal may be indicative of a full charge state. The method may include responding to the signal by controlling a corresponding one of the at least two switches to disconnect a corresponding one of the at least two Tx coils from the driver. For example, at least one of the external device and/or implantable device may include at least one temperature sensor configured for use to determine temperature events, which may be potentially harmful events. For example, the potentially-harmful temperature events may be defined based on the CEM43 dose threshold in standard ISO 14708-3:2017. The method may include responding to temperature events by temporarily disconnecting at least one Tx coil, for a duration such as a set time period or at least until the detected temperature event subsided. For example, the method may include determining coil alignment between a selected one of the at least two coils and a Rx coil in a corresponding one of the implantable devices. The driver may be used to drive the selected one of the at least two Tx coils (e.g., with a fixed voltage or a fixed current) to generate a fixed charge field. The signal may be indicative of the electrical energy transfer from the external device using the fixed charge field. The external device may be configured to determine coil alignment using the signal. For example, a fixed voltage may be generated so that the resulting coil current can be measured, as changes in the measured current indicate alignment or uplink data from implants as the loading on the coils change.

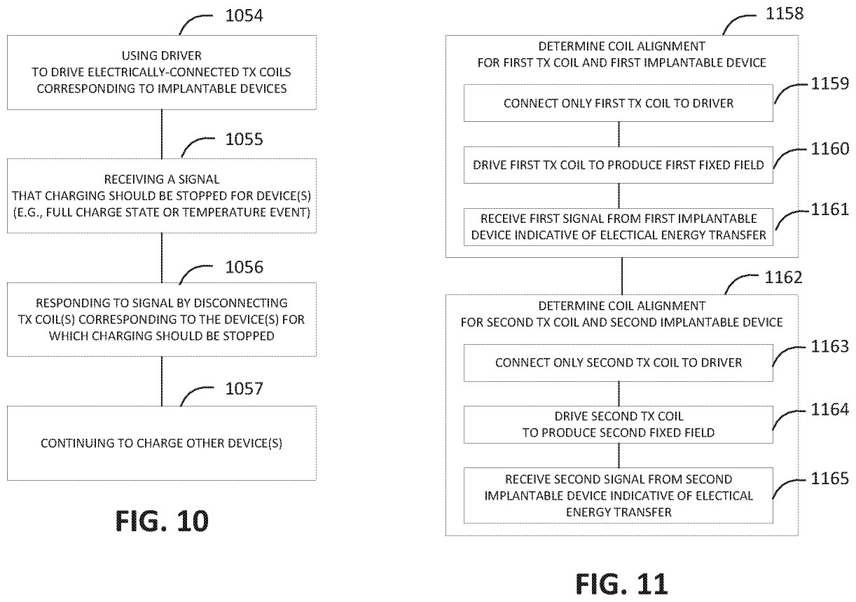

FIG. 10 illustrates, by way of example and not limitation, a method for charging two or more implantable devices. The method may be performed by an external device to recharge two or more implantable devices where the external device has a driver configured to drive two or more electrically-connected Tx coils corresponding to the two or more implantable devices. The subject matter may include using the driver to drive the two or more electrically-connected Tx coils to the driver to produce a charge field for recharging the two or more implanted devices 1054, receiving a signal that charging for one or more of the at least two implantable devices should be stopped 1055, responding to the signal by disconnecting one or more of the two or more Tx coils that correspond to the one or more of the at least two implantable devices for which charging should be stopped 1056, and continuing to charge one or more other devices from the at least two implantable devices 1057. The signal may indicate that the one or more of the at least two implantable devices are fully charged. The signal indicates that there is a temperature event associated with the charging of the one or more of the at least two implantable devices. The temperature event may be determined using at least one temperature sensor on the external device. The temperature event may be determined using at least one temperature sensor on the one or more of the at least two implantable devices. The one or more of the two or more Tx coils may be temporarily disconnected in response to the temperature event until the temperature event is over.

For example, the system may perform a recharge session by identifying the number of implant devices to charge during initiation of a recharge session based on the current charge state of each implant device, isolating the headsets that correspond to any implant devices that are fully charged. A charge field may be applied and regulated by measuring the rectified DC voltage derived from the induced voltage on the Rx coil, designated as signal PWRIN, from each implant device that is currently being recharged, and adjusting the PWRIN signal based on the maximum value reported from all of the implant devices (e.g., decrease the charge field if PWRIN is less than 7 V or increasing the charge field if PWRIN is greater than or equal to 7V). During the recharge session, the system may continuously perform the following checks: stop charging an IPG temporarily when an over-temperature condition occurs while still charging the remaining IPGs. Resume charging when the over-temperature condition subsides; and stop charging an IPG when an IPG has completed charging while still charging the remaining IPGs. The recharge session may be finished when all IPOs have completed charging.

FIG. 11 illustrates, by way of example and not limitation, a method for determining coil alignment with implantable devices. The method may be performed by an external device having a driver configured to drive at least a first Tx coil and a second Tx coil. The subject matter may include determining coil alignment between the first Tx coil and the first implantable device 1158, including connecting the first Tx coil to the driver 1159, using the driver to drive the first Tx coil to produce a first fixed charge field 1160, and receiving a first signal from the first implantable device indicative of electrical energy transfer from the external device 1161. The method may include determining coil alignment between the second Tx coil and the second implantable device 1162, including connecting the second Tx coil to the driver 1163, using the driver to drive the second Tx coil to produce a second fixed charge field 1164, and receiving a second signal from the second implantable device indicative of electrical energy transfer from the external device 1165. The coil alignment between the first Tx coil and the first implantable device may be determined when the coil alignment between the second Tx coil and the second implantable device is determined. The coil alignment between the first Tx coil and the first implantable device may be determined at a different time than when the coil alignment between the second Tx coil and the second implantable device is determined.

For example, the system may perform a measure of alignment for an implant device and corresponding headset coil by isolating the specific headset coil, commanding the implant device to measure the PWRIN signal when a charge field is applied, applying a fixed charge field to the headset corresponding to the implant device, communicating with the implant device to read the measured PWRIN signal, determining the level of alignment (e.g., a numeric or binary value) based on the PWRIN signal. Coil alignment information may be communicated to a user using the external device. For example, a user interface may include text, color and/or sound or other status indicators that indicate if the coils are aligned. Some embodiments may provide suggested actions for moving the headset coils (Tx coils) into alignment with the implanted coils (Rx coils).

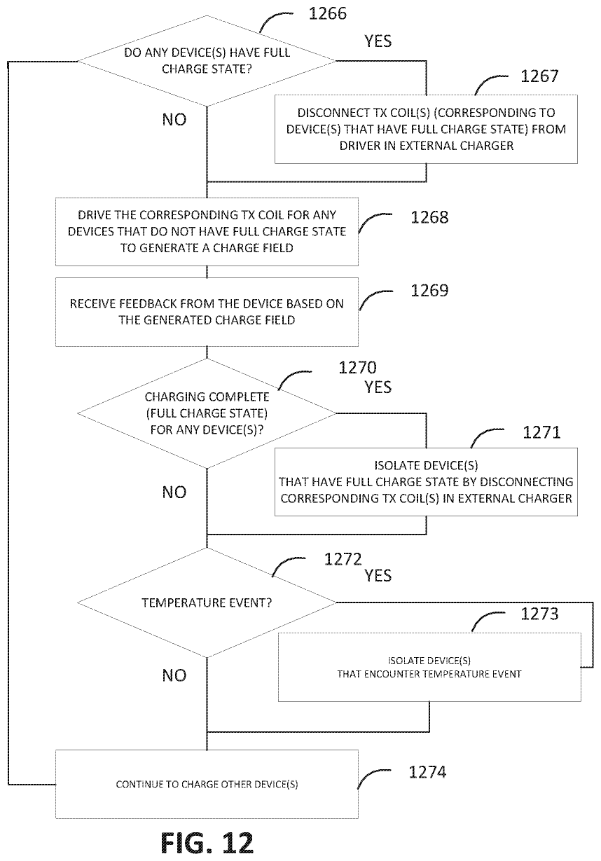

FIG. 12 illustrates, by way of example and not limitation, a method for charging two or more implantable devices while monitoring and accommodating temperature events. The method may include determining whether any device(s) have a full charge state at 1266. If there are device(s) with a full charge state, then the method may proceed to 1267 to disconnect the Tx coil(s) that correspond to device(s) the have a full charge state from the driver in the external charger. At 1268, the process drives the corresponding Tx coil for any devices that do not have full charge state to generate a charge field. Feedback may be received from the device based on the generated charge field 1269. By way of example, the feedback may be based on the received energy at the device (e.g., PWRIN). At 1270, the method may include determining whether charge is complete (e.g., “full charge state”) for any of the device(s). If charging is complete for any of the devices, then the process may proceed to 1271 to isolated the device(s) that have a full charge state by disconnecting corresponding Tx coil(s) in the external charger. At 1272, the method may include determining whether there has been a temperature event. The temperature event may be based on detected temperatures in the external device and /or the implantable device(s). If there has been a temperature event, then the process may proceed to 1273 to isolate device(s) that encounter the temperature event. Thus, for example, charging may be temporally stopped to allow the device or tissue interface to cool before resuming charging. At 1274, the process may proceed to continue to charge other device(s) in the system, returning to 1266.

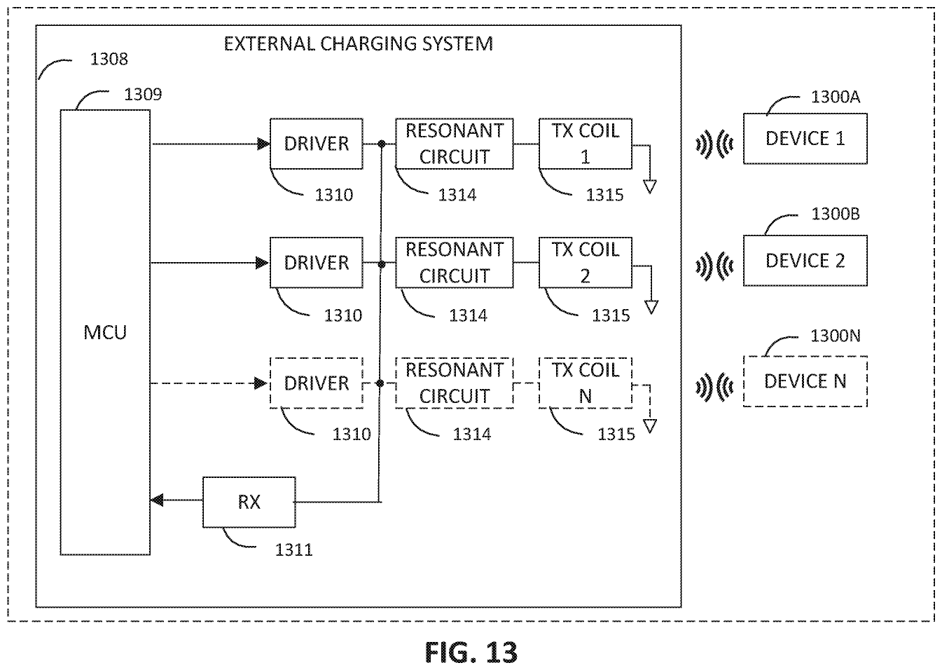

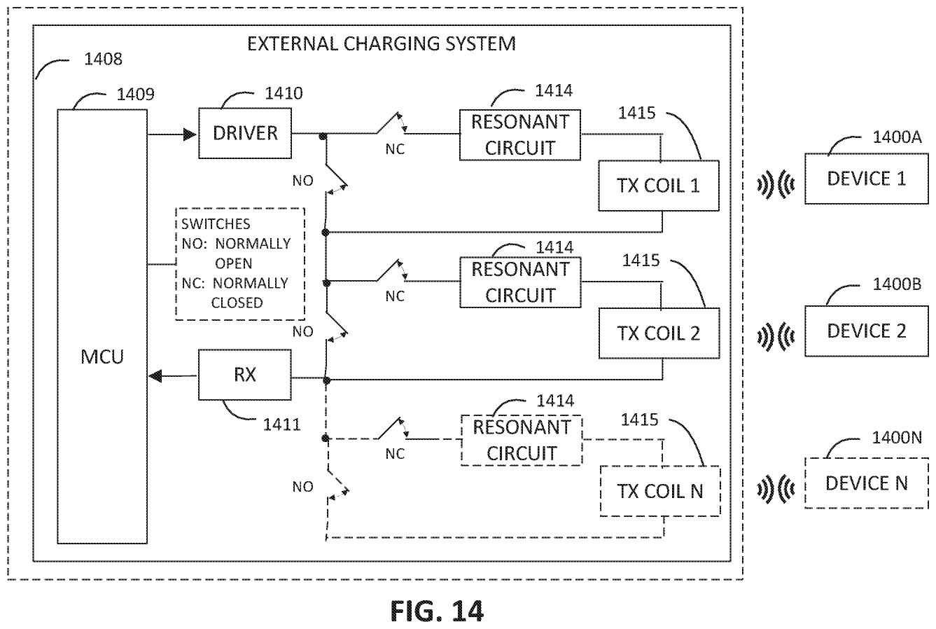

The embodiments of the external charging system described above used parallel coil circuit branches selectively connected to a single driver via switches controlled by a controller to individually control whether Tx coils are driven (e.g., FIG. 3). Other embodiments configured to individually control whether Tx coils are driven are illustrated in FIGS. 13-14. FIG. 13, for example, illustrates separate drivers, individually controlled by a controller, to drive separate Tx coil. The external charging system 1308 is configured to wirelessly charge at least two devices, and may be configured to wireless charge more than two devices (e.g., N devices), illustrated as Device 1 1300A, Device 2 1300B and Device N (1300N). The external charging system 1308 may include a controller 1309 (e.g., microcontroller or MCU), and a receiver circuit, or receiver 1311. The external charging system 1308 may further include at least two branches of circuit elements, where each branch includes a resonant circuit 1314 and a Tx coil 1315. The devices may not be “paired” to the Tx coils, but rather any of the Tx coils may be positioned and used to wireless charge any of the devices. That is, they may be interchangeable and do not have to be paired. The external charging system 1308 may have a dedicated driver 1310 for each branch. The controller 1309 may be configured to selectively control each of the drivers 1310 to control which of the Tx coils 1315 are driven. The receiver 1311 may be electrically connected to each resonant circuit to receive uplink communications. The resonant circuit 1314 creates a resonant frequency close to the drive frequency to maximize efficiency, and may also provide matching impedance to a cable such as a 75 Ohm cable. This design avoids using switches 313 as used in the embodiment illustrated in FIG. 3. Switches, such as relays, may be relatively big and may also have lifetimes that are shorter than desirable. The driver size may be less than the size of the switches. However, it is simpler to have one driver as the drivers may interfere with each other.

FIG. 14, for example, illustrates series-connected coils circuit branches selectively connected to a single driver via switches controlled by a controller. The external charging system 1408 is configured to wirelessly charge at least two devices, and may be configured to wireless charge more than two devices (e.g., N devices), illustrated as Device 1 1400A, Device 2 1400B and Device N (1400N). The external charging system 1408 may include a controller 1409 (e.g., microcontroller or MCU), and a driver 1410 The external charging system 1408 may further include at least two branches of circuit elements, where each branch includes a resonant circuit 1414 and a Tx coil 1415. The devices may not be “paired” to the Tx coils, but rather any of the Tx coils may be positioned and used to wireless charge any of the devices. That is, they may be interchangeable and do not have to be paired. Each coil branch contains two switches which may be controlled by the controller 1409: a normally closed (NC) switch in series with the branch, and a normally open (NO) switch in parallel with the branch. To remove a coil branch from the network, the controller 1409 may close the corresponding NO switch and open the corresponding NC switch. The system always needs to have at least one branch connected, or more specifically, all of the NO switches cannot be closed at the same time or the power amplifier will be shorted to ground. The illustrated system includes a receiver circuit or receiver 1411. The controller 1409 is capable to selectively connect the transmit coils 1415 to the driver 1410 and receiver 1411. An advantage of this system is that the changes in current created by one implant during uplink are not only reflected to the headset and external but also to all of the other implants in the network simultaneously allowing for direct implant-to-implant communication. This could have an application in a medical implant system consisting of multiple types of implanted devices such as several individual sensor implants distributed throughout the body and one or more therapy delivering implants that make decisions based on that data. All of the implanted devices in such a system would require very little to no internal power for communication and would be communicating over a proximity secured near field network.

Benefits of using coils that can be independently isolated include providing communication and charging for all implanted devices in the system independent of the modes of operation of the other implant devices. The system may also charge both implanted devices simultaneously when conditions permit to minimize the charging time. The system provides a mechanism for providing direct/active feedback from the implant device for a measure of alignment, compared to other approaches use passive measurements (Tx coil current, or reflective impedance) that do not utilize feedback from the implant.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A system, comprising:
at least two coil circuit branches, each of the at least two coil circuit branches including a transmit (Tx) coil and a switch, wherein the at least two coil circuit branches are connected in parallel;
a driver connected to the at least two coil circuit branches that are connected in parallel, wherein the driver is configured to drive the Tx coils in the at least two coil circuit branches; and
a controller configured to independently control the switch in each of the at least two coil circuit branches to independently control whether the driver is electrically connected to drive the Tx coil in the corresponding coil circuit branch.

2. The system of claim 1, wherein, for each of the at least two coil circuit branches, the switch is connected in series with the Tx coil.

3. The system of claim 1, wherein each of the at least two coil circuit branches further comprises a resonant circuit.

4. The system of claim 3, wherein, for each of the at least two coil circuit branches, the switch, the resonant circuit and the Tx coil are connected in series.

5. The system of claim 1, further comprising at least two implantable devices configured to be charged using the at least two coil circuit branches.

6. The system of claim 5, wherein the system is configured to independently control the switch in each of the at least two coil circuit branches to independently control which one or more of the at least two implantable devices are being charged.

7. The system of claim 6, wherein, when charging any one or more of the at least two implantable devices, the system is configured to determine and respond to an over-temperature event and respond to the determined over-temperature event by controlling the switch to prevent the driver from driving one or more of the Tx coils until the system determines that the over-temperature event has ended.

8. The system of claim 6, wherein the system is configured to simultaneously charge the at least two implantable devices, to determine when charging for one of the at least two implantable devices is completed, to control the switch to prevent the driver from driving the Tx coil for one of the at least two coil circuit branches that corresponds to the one of the at least two implantable devices with charging completed, and continuing to charge one or more other devices from the at least two implantable devices.

9. The system of claim 1, wherein the controller is configured to receive feedback from each of the at least two implantable devices using uplink communication, and to detect coil alignment using the received feedback.

10. The system of claim 1 wherein the controller is configured to receive feedback via an uplink from each of the at least two implantable devices, and to detect charging status using the received feedback.

11. The system of claim 1, wherein the controller is configured to receive feedback via an uplink from each of the at least two implantable devices, the received feedback is based on a PWRIN signal in each of the least two implantable devices, and the PWRIN signal corresponds to a voltage level of a receive coil energy.

12. The system of claim 1, wherein the at least two coil branches include a first coil branch corresponding to a first implantable device and a second coil branch corresponding to a second implantable device, and the controller is configured to determine coil alignment by:
connecting the first coil branch to the driver, using the driver to drive the first coil branch to produce a first fixed charge field, and receiving a first signal from the first implantable device indicative of electrical energy transfer from the external device; and
connecting the second coil branch to the driver, using the driver to drive the second coil branch to produce a second fixed charge field, and receiving a second signal from the second implantable device indicative of electrical energy transfer from the external device.

13. The system of claim 1, wherein the controller is configured to implement a recharge session by:
using the driver to drive the two or more coil circuit branches to produce a charge field for recharging a corresponding two or more implantable devices;
receiving a signal that charging for one or more of the at least two implantable devices should be stopped;
responding to the signal by disconnecting one or more of the two or more coil circuit branches that correspond to the one or more of the at least two implantable devices for which charging should be stopped; and
continuing to charge one or more other devices from the at least two implantable devices.

14. The system of claim 13, wherein the signal indicates that the one or more of the at least two implantable devices are fully charged.

15. The system of claim 13, wherein the signal indicates that there is a temperature event associated with the charging of the one or more of the at least two implantable devices.

16. The system of claim 15, wherein the temperature event is determined using at least one temperature sensor on the external device.

17. The system of claim 15, wherein the temperature event is determined using at least one temperature sensor on the one or more of the at least two implantable devices.

18. The system of claim 15, wherein the one or more of the at least two coil circuit branches are temporarily disconnected in response to the temperature event until the temperature event is over.

19. A method performed using an external device having a driver and at least two switches corresponding to at least two transmit (Tx) coils, wherein the at least two Tx coils correspond to at least two implantable devices, wherein each of the at least two switches are configured for electrically connecting a corresponding one of the at least two Tx coils to the driver, the method comprising:
independently controlling each of the at least two switches to cause one or more of the at least two Tx coils to be electrically-connected coils to the driver; and
using the driver to drive the one or more electrically-connected Tx coils to the driver.

20. The method of claim 19, further comprising receiving a signal from each of one or more implantable devices corresponding to the one or more electrically-connected Tx coils, wherein the received signal is indicative of electrical energy transfer from the external device to the corresponding implantable device.

21. The method of claim 20, further comprising recharging one or more of the at least two implantable devices, wherein the received signal is indicative of a full charge state, the method further comprising responding to the signal by controlling a corresponding one of the at least two switches to disconnect a corresponding one of the at least two Tx coils from the driver.

22. The method of claim 20, further comprising determining coil alignment between a selected one of the at least two Tx coils and a receive (Rx) coil in a corresponding one of the implantable devices, wherein:
the using the driver to drive the one or more electrically-connected Tx coils includes driving the selected one of the at least two Tx coils to generate a fixed charge field;
the signal is indicative of the electrical energy transfer from the external device using the fixed charge field; and
the external device is configured to determine coil alignment using the signal.

23. A method performed by an external device to recharge two or more implantable devices, the external device having a driver configured to drive two or more electrically-connected transmit (Tx) coils corresponding to the two or more implantable devices, the method comprising:
using the driver to drive the two or more electrically-connected Tx coils to the driver to produce a charge field for recharging the two or more implanted devices;
receiving a signal that charging for one or more of the at least two implantable devices should be stopped;
responding to the signal by disconnecting one or more of the two or more Tx coils that correspond to the one or more of the at least two implantable devices for which charging should be stopped; and
continuing to charge one or more other devices from the at least two implantable devices.

24. The method of claim 23, wherein the signal indicates that the one or more of the at least two implantable devices are fully charged.

25. The method of claim 23, wherein the signal indicates that there is a temperature event associated with the charging of the one or more of the at least two implantable devices.

26. The method of claim 25, wherein the temperature event is determined using at least one temperature sensor on the external device.

27. The method of claim 25, wherein the temperature event is determined using at least one temperature sensor on the one or more of the at least two implantable devices.

28. The method of claim 25, wherein the one or more of the two or more Tx coils is temporarily disconnected in response to the temperature event until the temperature event is over.

29. A method performed by an external device, the external device having a driver configured to drive at least a first transmit (Tx) coil and a second Tx coil, the method including:
determining coil alignment between the first Tx coil and a receive (Rx) coil for a first implantable device, including:
connecting the first Tx coil to the driver;
using the driver to drive the first Tx coil to produce a first fixed charge field; and
receiving a first signal from the first implantable device indicative of electrical energy transfer from the external device;
determining coil alignment between the second Tx coil and a Rx coil for a second implantable device, including:
connecting the second Tx coil to the driver;
using the driver to drive the second Tx coil to produce a second fixed charge field; and
receiving a second signal from the second implantable device indicative of electrical energy transfer from the external device.

30. The method of claim 29, wherein the coil alignment between the first Tx coil and the Rx coil for the first implantable device is determined when the coil alignment between the second Tx coil and the Rx coil for the second implantable device is determined.

31. The method of claim 29, wherein the coil alignment between the first Tx coil and the Rx coil for the first implantable device is determined at a different time than when the coil alignment between the second Tx coil and the Rx coil for the second implantable device is determined.

32. A system, comprising:
at least two coil circuit branches, each of the at least two coil circuit branches including a transmit (Tx) coil;
at least one driver connected to the at least two coil circuit branches to drive the Tx coils; and
a controller configured to independently control whether individual Tx coils are driven.

33. The system of claim 32, wherein the at least one driver includes a dedicated driver for each one of the at least two coil circuit branches, wherein the controller is configured to independently control the drivers to independently control whether individual Tx coils are driven.

34. The system of claim 32, wherein each of the at least two coil circuit branches includes a controller-controlled switch, and the at least two coil circuit branches are in parallel.

35. The system of claim 32, wherein the at least two coil circuit branches are configured to be connected in a series connected circuit, and the controller is configured to independently control whether individual Tx coils are driven by controlling whether each of the at least two coil circuit branches are in the series connected circuit.
